(19) United States
(12) Patent Application Publication
(10) Pub. No.: US 2025/0074865 A1
(43) Pub. Date: Mar. 6, 2025

(54) METHOD FOR THE MANUFACTURE OR CONVERSION OF ALKANOLAMINES

(71) Applicant: BASF SE, Ludwigshafen am Rhein (DE)

(72) Inventors: Michael JOEDECKE, Ludwigshafen am Rhein (DE); Franz BORGEL, Ludwigshafen am Rhein (DE); Christian GRUENANGER, Ludwigshafen am Rhein (DE); Markus HILLER, Ludwigshafen am Rhein (DE)

(21) Appl. No.: 18/728,111

(22) PCT Filed: Jan. 3, 2023
(86) PCT No.: PCT/EP2023/050035

§ 371 Date: Jul. 11, 2024

(30) Foreign Application Priority Data
Jan. 14, 2022 (EP) EP 22151521.6

(51) Int. Cl. C07C 213/02

(52) U.S. Cl. CPC C07C 213/02

(57) ABSTRACT

A process for the manufacture of alkanolamines and/or a process for the conversion of alkanolamines wherein the process is performed in one or equipment items, wherein one or more steel parts of the one or more equipment items, which have one or more surfaces in contact with an alkanolamine, are made of duplex steel and Use of duplex stainless steel as a construction material for parts of an equipment item used in an alkanolamine and/or alkyleneamine manufacturing process or separation process, which have one or more surfaces in contact with alkyleneamines and/or alkanolamines and the use of duplex steel as a construction material for steel parts of a equipment items used in an alkanolamine manufacturing or conversion process.

The present invention relates to a process for the manufacture of alkanolamines and a process for the conversion of alkanolamines wherein the process is performed in one or more equipment items, wherein one or more steel parts of the one or more equipment items, which have one or more surfaces in contact with the alkanolamine, are made of duplex steel. The present invention also relates to the use of duplex steel as a constructing material in an alkanolamine manufacturing or conversion process.

### Examples of Alkanolamines are Ethanolamines and Propanolamines

Ethanolamines and propanolamines are used as an intermediate in the production of surfactants and also find uses as catalysts for polyurethanes, intermediates for agrochemicals and pharmaceuticals, cosmetics, corrosion inhibitors, cement additives and lubricants. Ethanolamines and propanolamines are also used in gas treatment.

Alkanolamines are also converted to alkylene amines.

The most important representatives of this group of compounds are ethylene amines, such as ethylenediamine, diethylenetriamine, triethylenetetramine, piperazine, aminoethylpiperazine, and propylene amines, such as propylene-1,2-diamine and propylene-1,3-diamine.

Ethylenediamine is used predominantly as an intermediate for the production of bleach activators, crop protection agents, pharmaceuticals, lubricants, textile resins, polyamides, paper auxiliaries, gasoline additives and many other substances.

Propylene-1,2-diamine is an important intermediate for crop protection agents and used as a fuel and lubricant additive

Propylene-1,3-diamine is used for the production of a textile finishing agent or a chelating agent.

Other commercially relevant alkanolamines and alkylene amines are amino diglycol (ADG), polyglycol amines, polyproyleneglycolamines and polyethlyeneglycolamines.

Alkanolamine manufacturing and conversion processes generally have in common that they are conducted at higher temperatures and pressures and in the presence of ammonia or another aminating agent. Often hydrogen is also present in the reaction mixture.

Manufacturing and separation equipment items—such as reactors, columns, tanks, pipes, heat exchangers, condensers—used under such conditions need to fulfil different requirements, such as mechanical stability, temperature stability, corrosion resistance, and resistance to wear.

In addition, equipment items should be made of construction materials having a good processability. Further they should show a good resistance to withstand the conditions of the production process.

In the literature, the choice of material for equipment used in an alkanolamine manufacturing or conversion process is seldom discussed.

Most references in the literature only refer to the general use of stainless steel as a reactor material.

Some alkanolamines and alkylene amines are used as anticorrosion agents. This seems to imply that the choice of material for manufacturing and/or separation equipment items used for the manufacturing and/or separation of alkanolamines and/or alkylene amines is not in particular critical.

Despite of this, it was now found that equipment items made of steel used in alkanolamine manufacturing or conversion processes are subject to corrosion and therefore to more frequent repairs and replacements. This significantly reduces the uptime of an alkanolamine production or conversion plant due to a higher repair and replacement frequency. Frequent repairs and replacements also increase the overall production costs.

It was therefore object of the present invention to find a material for equipment items, which are used for the manufacture, conversion, or separation of alkanolamines, which is highly resistant to the conditions of an alkanolamine manufacture, conversion or separation process.

Especially, it was the object of the invention to find a material which is temperature and pressure-stable and which shows a good resistance to corrosion and wear.

In doing so, it was intended to prolong the service life of the equipment before replacement or repair.

The downtime of the process, due to repairs of equipment or due to material failure or material weakening was also intended to be reduced.

It was also intended to increase the operational safety of the equipment used in such production or separation processes, to avoid and prevent failure of critical equipment, which is required for the safe operation of the production and separation processes.

The construction material should show a good mechanical stability allowing for equipment design using less material compared to conventional steel equipment. The reduction of the required amount of construction materials is especially beneficial in view of increasing shortage of resources, such as steel.

The object of the present invention was therefore solved by a process according to claim 1 and the use of duplex steel according to claims 1 and 17.

Surprisingly, it was found that the material selection for equipment used for the manufacture or conversion of alkanolamines is not arbitrary and that advantages may be obtained using selected reactor materials for the conditions encountered during an alkanolamine manufacturing, conversion, or separation process.

### Alkanolamines

The process of the present invention is a process for the manufacture or conversion of alkanolamines.

Alkanolamines are a class of compounds comprising both hydroxyl- and amino functional groups on an alkane or alkoxyalkane backbone.

Preferred alkanolamines are ethanolamines, propanolamines and polyglycol monoamines.

Preferred ethanolamines are compounds selected from the group consisting of
monoethanolamine (MEOA)
diethanolamine (DEOA)
triethanolamine (TEOA)
N-methyl-ethanolamine (NMEA)
N-methyl-diethanolamine (NMDEA)
dimethylethanolamine (DMEA)
diethylethanolamine (DEEA)
n-butyl-ethanolamine
n-butyl-diethanolamine
dibutyl-ethanolamine
cyclohexyl-ethanolamine
cyclohexyl-diethanolamine
1-(2-hydroxyethylpiperazine
4-(2-Hydoxyethyl)-morpholine
Hydroxyethyl aniline
Ethyl-hydroxyethyl-aniline
N-ethyl ethanolamine
n-propyl ethanolamine
hydroxyethyl piperidine
di-hydroxyethyl aniline
tert-butyl ethanolamine
tert-butyl diethanolamine

More preferred ethanolamines are MEOA, DEOA, TEOA, NMEA, NMDEA, DMEA and DEEA.

Preferred propanol amines are compounds selected from the group consisting of:
2-amino-1-propanol (2-AP)
1-amino-2-propanol (1-AP)
Isopropanol amine (IPA) (mixture of 2-amino-1-propanol and 1-amino-2-propanol)
di-isopropanol amine (DIPA)
tri-isopropanol amine (TIPA)
mono-methyl-iso-propanolamine
methyl-di-isopropanol amine
dimethyl-iso-propanol amine
di-ethyl-isopropanol amine
di-butyl-isopropanol amine
cyclohexyl-isopropanol amine
cyclohexyl-di-isopropanol amine
cyclooctyl-isopropanol amine
cyclooctyl-di-isopropanol amine
aminoethyl-isopropanol amine
1-(2-hydroxypropyl)-piperazine
1,4-bis(2-hydroxypropyl)-piperazine
4-(2-Hydroxypropyl)-morpholine
N,N,N′N′-tetrakis-(2-hydroxypropyl)-ethylenediamine

More preferred propanolamines are 2-AP, 1-AP, IPA, DIPA and TIPA.

Most preferred propanolamines are propanolamines obtained by reaction of propylene oxide with ammonia, namely 1-AP, 2-AP, IPA, DIPA and TIPA.

Another preferred alkanolamine is amino glycol amine (ADG).

Preferred polyglycol monoamines are obtained from the reaction of polyglycols, such polyethylene glycols or polypropylene glycols, with a number averaged molecular weight of 200 to 5000.

### Alkylene Amines

Alkylene amines are compounds comprising at least two amine functional groups (primary, secondary or tertiary) on an alkane or alkoxyalkane backbone.

Preferred alkylene amines are ethylene amines, propylene amines and polyglycol diamines.

More preferred alkylene amines are alkylene amine compounds derived from MEOA (ethylene amines), 1,2 Aminopropanol (1,2-proplyene amines), 1,3-aminopropanol (1,3-propylene amines) and polyether-mono-amines (polyetheramines).

Most preferred ethylene amines are ethylene diamine, diethylene triamine, triethylene tetramine, tetraethylene pentamine, pentaethylene hexamine, piperazine and aminoethyl piperazine.

Most preferred 1,2-propylene amines are 1,2-propylene diamine, 1,2 dipropylene triamine, 1,2-tripropylene tetramine, 1,2-tetrapropylene pentamine and 1,2-pentrapropylene hexamine.

Most preferred 1,3-propylene amines are 1,3-propylene diamine, 1, dipropylene triamine, 1,3-tripropylene tetramine, 1,3-tetrapropylene pentamine and 1,3-pentrapropylene hexamine.

Most preferred polyglycol diamines are polyglycol diamines of formula (I)

R¹R²N—(CHR³—CH₂—O)_{I}—CH₂—CHR—OH (I),

wherein
R¹ and R² have the meaning as specified below,
R³ is H, methyl or ethyl, and
I is an integer from 2 to 70, preferably 2 to 10 and most preferably 2 to 6.

### Alkanolamine Manufacturing Process and Alkanolamine Conversion Process

Processes for the manufacture and conversion of alkanolamines are known in the art. The most important routes are summarized below.

Alkanolamines may be prepared by conversion of an alkylene oxide (Alkylene Oxide Process), a polyol (Polyol Amination Process), a hydroxy ketone or a hydroxy aldehyde (Reductive Amination Process) with an aminating agent.

Alkanolamines may further be converted with an aminating agent to the corresponding alkylene amines (Alkanolamine Amination Process).

The aminating agent is a compound selected from the group consisting of ammonia, primary and secondary amines.

Preferably, the aminating agent is a compound of formula (II)

NHR¹R² (II);

in which
R¹ and R² are independently hydrogen,
C₁-C₂₀-alkyl, in particularly methyl, ethyl, n-propyl, isopropyl, n-butyl, iso-butyl, sec-butyl, n-pentyl, iso-pentyl, sec-pentyl, neo-pentyl, 1,2-dimethylpropyl, n-hexyl, iso-hexyl, sec-hexyl, n-heptyl, iso-heptyl, n-octyl, 2-ethylhexyl, n-decyl, 2n-propyl-n-heptyl, n-tridecyl, 2-n-butyl-n-nonyl and 3-n-butyl-n-nonyl, more preferably C₁-C₄-alkyl, in particularly methyl, ethyl, n-propyl, iso-propyl, n-butyl, iso-butyl and sec-butyl;
C₃-C₁₂-cycloalkyl, in particularly cyclo-pentyl, cyclo-hexyl, cyclo-heptyl, cyclo-octyl, preferably C₅-C₈-cycloalkyl, in particularly cyclo-pentyl, cyclo-hexyl, cyclo-heptyl and cyclo-octyl, R¹ and R² may jointly are —(C)ⱼ—X—(C)ₖ—, wherein j and k are integers from 1 to 4, in particularly —(CH₂)₃—, —(CH₂)₄—, —(CH₂)₅—, —(CH₂)₆—, —(CH₂)₇—, —(CH₂)—O—(CH₂)₂—, —(CH₂)—NR⁵—(CH₂)₂—, —(CH₂)—CHR⁵—(CH₂)₂—, —(CH₂)₂—O—(CH₂)₂—, —(CH₂)₂—NR⁵—(CH₂)₂—, —(CH₂)₂—CHR⁵—(CH₂)₂—, —CH₂—O—(CH₂)₃—, —CH₂—NR⁵—(CH₂)₃—, and —CH₂—CHR₅—(CH₂)₃.

Most preferred aminating agents are aminating agents selected from the group consisting of ammonia, methyl amine, dimethyl amine, ethyl amine, diethyl amine, n-propyl amine, iso-propyl amine, di-n-propyl amine, di-iso-propyl amine, n-butyl amine, iso-butyl amine, sec-butyl-amine, di-n-butyl amine, di-iso-butyl amine, di-sec-butyl-amine, n-pentyl-amine, n-hexyl amine, cyclo-hexyl and cyclo-octyl amine.

In particularly preferred aminating agents are aminating agents selected from the group consisting of ammonia, methyl amine, ethyl amine, n-propyl amine, iso-propyl amine, n-butyl amine, iso-butyl amine, sec-butyl amine and cyclohexyl amine.

Further processes for the manufacture or conversion of alkanolamines are the Amino Acid Process and the Urea Process (both specified below).

### DETAILED PROCESS DESCRIPTION

The Alkylene Oxide Process, the Polyol Amination Process, the Reductive Amination Process, the Amino Acid Process and the Urea Process will be described in further detail below:

### Alkylene Oxide Process

In a preferred embodiment, alkanolamines are obtained by conversion of an alkylene oxide with an aminating agent (“Alkylene Oxide Process”).

Preferred alkylene oxides are ethylene oxide, propylene oxide and butylene oxide.

Most preferred alkylene oxides are ethylene oxide and propylene oxide.

The Alkylene Oxide Process is the most preferred process for the manufacture of ethanol amines and 1,2-propanol amines

The most preferred ethanol amines manufactured by the Alkylene Oxide Process are monoethanolamine (MEOA), diethanolamine (DEOA), triethanolamine (TEOA), N-methyl-ethanolamine (NMEA), N-methyl-diethanolamine (NMDEA), dimethylethanolamine (DMEA) and diethylethanolamine (DEEA).

Such ethanol amines are obtained by the reaction of ethylene oxide with the corresponding aminating agent, in particular ammonia and the corresponding alkylamines and dialkylamiens, such as methylamine, dimethylamine, ethylamine and diethylamine.

The most preferred 1,2-propanol amines which may be manufactured by the Alkylene Oxide Process are 2-amino-1-propanol (2-AP), 1-amino-2-propanol (1-AP), isopropanol amine (IPA) (mixture of 2-amino-1-propanol and 1-amino-2-propanol), di-isopropanol amine (DIPA) and tri-isopropanol amine (TIPA).

Such 1,2-propanol amines may be obtained by the reaction of propylene oxide with the corresponding aminating agent, in particularly ammonia or the corresponding alkylamines and dialkylamines.

An overview of the Alkylene Oxide Process can be found in the chapter “Ethanolamines and Propanolamines” in Ullmann's Encyclopedia of Industrial Chemistry, https://doi.org/10.1002/14356007).

According to this publication, ethanol amines are produced on an industrial scale by reaction of ethylene oxide with excess ammonia. The reaction is accelerated by water. The ammonia concentration is usually between 50 to 100%. Pressures are ordinarily in the range of up to 160 bars and temperatures up to 150° C. The molar excess of ammonia is usually up to 40 mol per mol ethylene oxide. The reaction yields monoethanolamine and diethanolamine and triethanolamines as side products. Unconsumed ammonia and water are usually separated from the products in a downstream purification process and recycled to the reactor. A process flow diagram of a typical ethylene oxide conversion is depicted in FIG. 2 of the afore-mentioned Ullmann chapter.

Further process variants can be found in the PERP report 01/02S2 “Ethanolamines” (2002) available from Nexant Chem Systems or in the Sulzer Technical review 3/2008 (https://www.sulzer.com/-/media/files/products/process-techology/processes-and-applications/2008_3_12_faessler_e.pdf?la=en).

Further details can also be found in the patent literature, such as U.S. Pat. Nos. 2,196,554, 3,697,598, 3,723,530, WO 2006/224417, WO 01/94290, EP1291339, DE1941859, EP1652207, DE1941859 und EP1652207. The aforementioned references are herewith incorporated by reference.

The manufacture of isopropanol amines is similar to the production of ethanol amines, except that propylene oxide is used as the alkylene oxide component instead of ethylene oxide (see Chapters 4.2 and 5.2 in the aforementioned Ullmann article).

### Polyol Amination Process

In a further preferred embodiment, alkanol amines are manufactured by conversion of a polyol with an aminating agent in the presence of an amination catalyst (Alcohol Amination Process).

Polyols are compounds comprising more than one hydroxyl group.

Preferred polyols are selected from the group consisting of
moethylene glycol,
diethylene glycol,
polyols of formula (2)
HO—CHR³—CH₂—(O—CHR³—CH₂)_{I}—OH, wherein R³ and I have the meaning given to the above, in particularly polyethylene glycols (PEG) and polypropylene glycols (PPG) and with a number averaged molecular weight of 200 g/mol to5000 g/mol, preferably of 200 g/mol to 500 g/mol,
1,2-propanediol, 1,3-propandiol, 2-methyl-1,3-propanediol, 1,4-butanediol, 1,2 butanediol, 1,3-butanediol, 1,5-pentandiol, 1,6-hexandiol, 1,7-heptanediol, 1,8-octanediol, 1,9-nonanediol, 1,10-decanediol, 1,11-undecanedio, 1,12-dodecanediol
glycerine, trimethylolpropane, neopentyl glycol, trimethylpentane diol.

Preferred aminating agents are the aminating agents referred to above.

Most preferred aminating in the context of the Polyol Amination Process are ammonia, methylamine, ethylamine, n-propylamine, di-isopropylamine, n-butylamine, sec-butylamine, iso-butylamine and tert-butylamine.

Detail relating to the manufacture of alkanolamines from polyols can be found in EP0382049, EP2234717, EP2225030, EP2225027 or EP0382049, all of which are herewith incorporated by reference.

In a particularly preferred embodiment, the Polyol Amination Process is a process for the conversion of
ethylene glycol with ammonia (MEG Amination Process),
diethylene glycol with ammonia (DEG Amination Process),
1,2-propanediol or 1,3-propanediol with ammonia (PDO Amination Process), or
polyglycols of formula (1), preferably PPG with a number molecular weight from 200 to 500 g/mol with ammonia (Polyglycol Amination Process).

The Polyol Amination Process can be seen as both a process for the manufacture of alkanolamines and as a process for the conversion of alkanolamines, because intermediary alkanolamines may react further to the corresponding alkylene amines. The product distribution between alkanolamines and alkylene amines dependent on the molar ratio of aminating agent to polyol. Excess of aminating agent usually promotes the formation of alkylene amines.

### MEG Process

A preferred Polyol Amination Process is the conversion of monoethylene glycol with ammonia (MEG Process) yielding a mixture of alkanolamines and ethylene amines.

The reaction of MEG with ammonia can be effected in the liquid phase or the gas phase. Gas phase reactions are disclosed, for example, in CN 102190588 and CN 102233272.

Preferably the conversion of MEG with ammonia is conducted in the liquid phase according to U.S. Pat. Nos. 4,111,840, 3,137,730, DE 1 72 268, WO 2007/093514, WO2007/093552, WO2018/224316, WO2018/224315, WO2018/224321 and WO2020/17085, all of which are hereby incorporated by reference.

Preferred amination catalysts for the MEG Process are:
the nickel-rhenium catalysts disclosed in U.S. Pat. No. 4,111,840,
the nickel-copper catalysts disclosed in U.S. Pat. No. 3,137,730,
the ruthenium-cobalt catalysts disclosed in WO2007093514,
the catalysts comprising cobalt, ruthenium and tin disclosed in WO2018224316,
the catalysts comprising tin and a further active metal disclosed in WO2018224315,
the impregnated catalysts disclosed in WO2018224321, or
the rhenium comprising catalysts disclosed o WO202017085.

The above cited documents disclosing suitable catalysts are explicitly incorporated by reference. Special preference is given to the catalysts disclosed in the examples of the above-referenced documents.

### DEG Process

A further preferred Polyol Amination Process is the conversion of diethylene glycol with ammonia (DEG Process) to yield a mixture of amino diglycol (ADG) and morpholine.

Details regarding the process can be found in WO2008006752, WO03051508, EP0695572, EP1937625 and EP2506966, all of which are herewith incorporated by reference.

### PDO Process

Yet another preferred Polyol Amination Process is the conversion of 1,2-propanediol or 1,3-propanediol with ammonia (DEG Process) in the presence of an amination catalyst to yield the corresponding propyleneamines and propanolamines. Process conditions and setup are similar to the conditions of the DEG Process.

### Polyglycol Amination Process

A further preferred Polyol Amination Process is the conversion of a polyglycols with ammonia to yield a mixture of polyglycol-monoamines and polyglycol-diamines (Polyglycol Amination Process).

Preferred polyglycols are polyols of formula (2), in particularly polypropylene glycols or polyethylene glycols with a number averaged molecular weight of 200 to 5000 g/mol, preferably 200 to 500 g/mol. Preferred propylene glycols have averaged number molecular weight of 230, 400, 2000 and 4000.

Details are found in EP696572, WO09092724, WO2008067857 and WO2016091643, which are herewith incorporated by reference.

### Reductive Amination Process

In still a further preferred embodiment, alkanolamines are manufactured by conversion of hydroxy-ketones or hydroxy aldehydes with an aminating agent in the presence of an amination catalyst (Reductive Amination Process).

Hydroxy ketones are compounds comprise at least one hydroxyl group and one keto-group.

Hydroxy aldehydes are aldehydes which comprise one or more hydroxyl groups.

Preferably hydroxy ketones and hydroxy aldehydes are selected from the group consisting of hydroxypivalaldehyde, dimethylolpropionaldehyde and dimethylolbutyraldehyde.

Further details are found in EP1487573, EP1999099 and EP2225029, all of which are herewith incorporated by reference.

### Glycolaldehyde Process

A particularly preferred embodiment of a Reductive Amination Process is the conversion of glycolaldehyde with an aminating agent, preferably ammonia, monoethanolamine or diethanolamine.

Details are disclosed in US20120259139, U.S. Pat. No. 8,772,548, WO2019193117, WO20200028322, WO2020249426, WO2020249427 and WO2020249428, all of which are herewith incorporated by reference.

### Amino Acid Process

A preferred route to alkanolamines is the hydrogenation of amino acids.

Preferred amino acids are serine, alanine and valine.

Details regarding the process conditions may be found in the article “Hydrogenation of Amino Acid Mixtures to Amino Alcohols”, by Pimparkar et al. (ind. Eng. Chem. Res. 2008, 47, 20, 7648-7653, Publication Date: Sep. 12, 2008, https://doi.org/10.1021/ie800351x), U.S. Pat. No. 6,310,254, EP0696575, WO99/38838 or US20070142648, all of which are herewith incorporated by reference.

### Alkanolamine Amination Process

A preferred embodiment of an alkanolamine conversion process is a process in which an alkanolamine is converted with an aminating agent to the corresponding di-or polyamines (Alkanol Amination Process).

### MEOA Process

A preferred Alkanol Amination Process is the conversion of monoethanolamine with ammonia to ethyleneamines in the presence of an amination catalyst (MEOA Process).

The reaction of MEOA and ammonia is described, for example, in U.S. Pat. No. 2,861,995, DE-A-1 172 268 and U.S. Pat. No. 3,112,318. An overview of the various process variants of the reaction of MEA with ammonia can be found, for example, in the PERP Report No. 138 “Alkyl-Amines”, SRI International, March 1981 (especially pages 81-99, 117), all of which are herewith incorporated by reference.

The reaction of MEOA with ammonia is preferably conducted in the presence of a transition metal catalyst at 150-250 bar and 160-210° C. or over a zeolite catalyst at 1-20 bar and 280-380° C.

Transition metal catalysts used with preference comprise Ni, Co, Cu, Ru, Re, Rh, Pd or Pt or a mixture of two or more of these metals on an oxidic support (e.g. Al₂O₃, TiO₂, ZrO₂, SiO₂).

Preferred zeolite catalysts are mordenites, faujasites and chabazites.

To achieve a maximum EDA selectivity, in the case of transition metal catalysis, a molar ratio of ammonia to MEOA of 6-20, preferably 8-15, is generally employed, and, in the case of zeolite catalysis, generally 20-80, preferably 30-50.

The MEOA conversion is generally kept between 10% and 80%, preferably 40-60%. In continuous operation, preferably, a catalyst space velocity in the range of 0.3-0.6 kg/(kg*h) (kg MEOA per kg cat. per hour) is established.

To maintain the catalyst activity, when metal catalysts are used, preference is given to additionally feeding 0.05-0.5% by weight (based on the MEOA+NH₃+H₂ reaction input) of hydrogen into the reactor.

### MPOA Process

A further preferred Alkanol Amination Process is the conversion of 1,2-propanol amine or 1,3 propanolamine with ammonia to the corresponding propylene amines in the presence of an amination catalyst (MPOA Process).

Reaction conditions are similar to those disclosed for the MEOA Process.

### Urea Process

A preferred alkanolamine conversion process is the so-called Urea Process in which an ethanol functional compound is converted with a primary amine in the presence of a carbon dioxide delivering agent. Details of such a process are disclosed in WO2017/137532, WO2019/030191 and WO2019/011710 which are also incorporated into this specification by reference.

### Disclaimer

The present invention disclaims any processes in which alkanolamines are used as absorbents for gas treating. Specifically, any reaction of an alkanolamine with an acid gas, such as CO₂, H₂S or COS, to a loaded absorbent and the regeneration of the loaded absorbent to alkanolamine is not considered to be an alkanolamine manufacturing or conversion process.

### Equipment Items

The process according to the invention is performed in one or equipment items.

Equipment items comprise all items, apparatuses, machinery or equipment which are required for the proper functioning of a plant in which alkanolamines are manufactured or converted and separated. The equipment items are usually summarized in a process flow sheet and listed in an equipment list.

Equipment items may comprise agitators, air filters, bin, blenders, blowers, centrifuges, compressors, condensers, conveyors, cooling towers, crushers, crystallizers, cyclone separators, decanters, dispersers, drums, dryers, evaporators, feeders, filters, furnaces, grinders, heat exchangers, kettles, kilns, mixers, ovens, pumps, reboilers, reactors, separator, spray disks, nozzles, tanks, towers, trays vacuum pumps, valves and pipes.

Such equipment items which are used for the conversion of one or more educts to the desired products are usually referred to as manufacturing equipment items.

Such equipment items which are used for the separation of one or more components from one or more manufacturing items in which the educts are at least partially converted to the desired products are usually referred to a separation equipment items.

### Parts of an Equipment Item

The equipment items can consist of a single part or it can consist of more than one part which are connected or adjoined to form the equipment item.

The one or more parts of an equipment item can be made of steel (“steel parts”) or they can be made of a different material (“non-steel parts”).

The one or more steel parts of an equipment item can have one or more surfaces which are in contact with an alkanolamines (“exposed steel parts”) or the one or more steel parts can have surfaces which are not in contact with an alkanolamine (non-exposed steel parts).

The present invention is directed to those parts of the equipment item which are usually made of steel. Accordingly, equipment item parts, which due to their purpose and requirements are usually made from a different material than steel, such as rubber or Teflon fittings, can still be used in the process according to the invention and are also not required to be made of duplex steel.

The present invention does not require that all steel parts of an equipment item are made of duplex steel. According to the invention, only one or more of the exposed steel parts of one or more equipment item are required to be made of duplex steel, whereas the non-exposed steel parts can be made of a non-duplex steel grade.

The present invention also does not require that not all exposed steel parts of all equipment items used in the manufacturing and/or separation process must be made of duplex steel.

The benefits of the present invention can already be obtained if at least one exposed steel part of one or more equipment item is made of duplex steel. However, the more exposed steel parts of an equipment item are made of duplex steel, the more benefit can be exploited.

If all exposed steel parts of an equipment item are made of duplex steel, it is avoided that one non-duplex steel part of an equipment item becomes a bottleneck for that particular equipment item.

If all exposed steel parts of all equipment items are made of duplex steel, it is avoided that one equipment item becomes the bottleneck for the manufacturing or separation process and problems encountered from the combination or joining of different material grades are usually avoided.

In a preferred embodiment, also one or more non-exposed steel parts of one or more equipment item are made of duplex steel. This has the advantage that problems are reduced, which result from the use of different material grades, such interface cracking or wear.

### Made of Duplex Steel

According to the invention, one or more steel parts of the one or more manufacturing and separation equipment items, which have one or more surfaces in contact with an alkanolamine, are made of duplex steel.

Within the meaning of this specification, the term “made of duplex steel” does not require that the entire steel part consists of duplex steel. The term “made of duplex steel” also comprises steel parts which are coated with duplex steel or which have inliners made of duplex steel.

In a preferred embodiment, the term “made of duplex steel” with respect to a steel part which has an exposed surface shall mean that the steel part is made entirely of duplex steel or consists of Duplex steel.

This has the advantage, that the manufacturing processes of equipment parts can be simplified and made more economical if only one steel grade is used when forming, spinning, pressing, bending or welding steel into the desired steel part

### Major and Ancillary Equipment Items

Equipment items may further be distinguished in major equipment items and ancillary equipment items.

Major equipment items are usually non-standard equipment items which are customized to meet the required process specifications.

Ancillary equipment items are usually equipment items which are used to support or assist major equipment items in meetings its functional duties. Ancillary equipment is more commonly available off-the shelf

### Major Equipment Items

The following lists of major equipment items are illustrative and not exhaustive because the major equipment items may vary with the exact implementation of the manufacturing or conversion process.

Also, the list of steel parts with an exposed surface of the major equipment is merely illustrative and not exhaustive, because also the parts can vary depending on the exact design of the major equipment item.

In a preferred embodiment, the one or more major equipment items for the alkanolamine manufacturing process and/or conversion process are one or more items selected from the group consisting of reactors, vessels, heat exchangers, condensers, agitators and columns.

Preferred reactors are tube reactors, fixed bed reactors and stirred tank reactors.

Preferred steel parts of reactors which have an exposed surface are preferably one or more parts selected from the group consisting of the reactor vessel or mantle, distributors, outlet collectors, support grids, quench pipes, support beams, diffusors, nozzles, baffles, center-pipes, scallops, trays, reactor lids, inlet heads and outlet heads.

As previously stated, it is preferred that all exposed steel parts of a major equipment item are made of duplex steel. If all exposed steel parts are made of duplex steel, it is avoided that a non-duplex part becomes the bottleneck in the major equipment which requires replacement or repair before the other duplex steel parts need to be replaced or repaired. Preferred vessels are flash vessels, storage vessels, buffer vessels and separation vessels.

Preferred steel parts of vessels which have an exposed surface are preferably one or more parts selected from the group consisting of the vessel container itself, the inlet, the outlet, de-entrainment meshes, vortex breakers, baffles and diffusers.

Preferred heat exchangers and condensers are tube shell heat exchangers, double pipe heat exchangers and plate heat exchangers.

Preferred steel parts of heat exchangers which have an exposed surface are preferably one or more parts selected from the group consisting of plates, covers, coils, baffles, tubes, shells, heads and sheets.

Preferred agitators are turbine agitators, anchor agitators, paddle agitators, propeller agitators, helical agitators.

Preferred steel parts of agitators which have an exposed surface are preferably one or more parts selected from the group consisting of shafts, impellers and impeller blades.

Preferred columns are bubble-cap tray columns, sieve tray columns, dual flow tray columns, valve tray columns, baffle tray columns or columns having random packings or structured packings.

Preferred steel parts of columns which have an exposed surface are preferably one or more parts selected from the group of shells or mantles, column heads, trays, packings, support grids, distributors, collectors, inlets and outlets.

A column is also preferably connected to a reboiler, such as a natural circulation evaporator or forced circulation evaporator. Alternatively, it is possible to use evaporators with a short residence time, such as falling-film evaporators, helical tube evaporators, wiped-film evaporators or a short-path evaporator.

Preferred steel parts of an reboiler which have an exposed surface are preferably one or more parts selects from the group of baffles, heating coils, heating plates, tube bundles, weirs, support plates, partition plates, shell, heads, outlet and inlets

### Ancillary Equipment

In a preferred embodiment, not only the steel parts with an exposed surface of a major equipment item are made of duplex steel, but also steel parts of ancillary equipment items which have an exposed surface.

Ancillary equipment items are preferably selected from the group consisting of pumps, compressors, pipes and valves.

In a more preferred embodiment also the fastening means which have an exposed surface are made of duplex steel.

Fastening means are means by which separate parts of an equipment item are connected to form the equipment item.

Preferably, fastening means are one or more means selected from the group of nuts, bolts, fittings, sealing rings, flanges and screws.

### Parts to be Made of Duplex Steel

As previously stated, the benefits of the present invention can already be exploited if at least one steel part of at least one equipment item is made of duplex steel.

However, the benefits of the present invention can be better exploited if one or more exposed steel parts of one or more major equipment items are made of duplex steel. Since major equipment items are usually tailored to the specific manufacturing or conversion process, it is more difficult to replace such items compared to equipment items which are standardized or available of the rack.

However, it is more preferably that all exposed steel parts of at least one major equipment item is made of duplex steel. This has the advantage that a non-duplex steel parts will not become a bottleneck for the stability of the entire equipment item.

It is also preferably, that all exposed steel parts of all major equipment items are made of duplex steel. This has the advantage that a major equipment item not made of duplex steel will become a bottleneck in the process.

Even more preferably, all steel parts (exposed and non-exposed steel parts) of at least on equipment item are made of duplex steel. In this case, disadvantages which many be incurred by the joining or combination of different material grades may be avoided.

It is further preferred that all steel parts of all major equipment items are made of duplex steel.

It is further preferred, that in addition to a high duplex steel content in the major equipment, as outlined above, also one or more exposed steel parts of one or more ancillary equipment items are made of duplex steel.

It is especially preferred that all exposed steel parts of one or more ancillary equipment items are made of duplex steel and more preferably all exposed steel parts of all ancillary equipment items are made of duplex steel. Even more preferably, all steel parts (exposed and non-exposed steel parts) of one or more, and still even more preferably of all ancillary equipment items are made of duplex steel.

In another preferred embodiment all exposed steel parts of all major and ancillary equipment items is made of duplex steel and preferably, all steel parts (exposed and non-exposed) of all ancillary and major equipment items is made of duplex steel.

### Hydrogen Exposure

In a most preferred embodiment, only those steel parts with an exposed surface are made of duplex steel which are additionally in contact with a liquid phase, wherein the hydrogen concentration in the liquid phase is 0.1 weight percent or less, preferably 0.05 weight percent or less and more preferably 0.01 weight percent or less. When the manufacturing or conversion process is conducted at hydrogen concentrations above the aforementioned limits, duplex steel is not the preferred material choice because the hydrogen may result in undesired hydrogen embrittlement of the duplex steel parts.

If the steel parts are additionally exposed to hydrogen above the afore-mentioned values, it is therefore preferred that such steel parts are made of conventional stainless steel having a good resistance to hydrogen embrittlement.

For alkanolamines manufacturing processes and alkanolamine conversion processes which are conducted in the presence of hydrogen above the afore-mentioned limits, it is therefore also preferred that only the separation equipment items downstream of manufacturing equipment items and after separation step which results in a sufficient reduction of the hydrogen concentration in the liquid phase, is made of duplex steel.

### Water Exposure

In a further preferred embodiment, only those exposed steel parts are made of duplex steel which are exposed to a liquid phase comprising water. Exposed to water within the meaning of the present invention means that the water concentration in the liquid phase comprising water and alkanolamines is 10 percent by weight or higher, preferably 20 percent by weight or higher and most preferably 30 percent by weight or higher. Aqueous alkanolamine solutions appear to be especially detrimental to the service life of manufacturing and separation equipment items.

### Temperature Exposure

In still another preferred embodiment, only those exposed steel parts are made of duplex steel which are exposed to a temperature of 30° C. or more, more preferably 40° C. or more, even more preferably 50° C. or more and most preferably 75° C. or more. If the exposed steel parts are also exposed to such temperatures, it has been found that the exposed steel parts are particularly prone to corrosion when not made of duplex steel.

### Pressure Exposure

In still another preferred embodiment, only those exposed steel parts are made of duplex steel which are exposed to a pressure of 10 mbar to 300 bar, more preferably 20 mbar to 250 bar and most preferably 40 to 200 bar.

### Corrosive Compounds

In still another preferred embodiment, the effluent obtained by the method according to one of the claims comprises at least one corrosive compound selected from the group consisting of (i) acids, selected from the group consisting of formic acid, acetic acid, oxalic acid, glycolic acid, glyoxilic acid, propionic acid and glycine, (ii) the corresponding ammonium salts of a such acids, (iii) carbamic acids, and (iv) ammonium carbamates.

It is preferred to conduct the methods and processes of the present invention in the absence of oxygen and carbon dioxide, that is under conditions in which the concentration of each oxygen and carbon dioxide is preferably 500 ppm or less, more preferably 400 ppm or less and most preferably 300 ppm or less. Surprisingly, it has been found that even though the method according to the present invention is preferably conducted in the absence of oxygen and carbon dioxide, the trace contaminants of oxygen and carbon dioxide within the process can lead to the formation of degradation products of alkanolamines, in particular when the alkanolamine is MEOA. These degradation products can be corrosive compounds. Corrosive compounds are preferable acids selected from the group consisting of formic acid, acetic acid, propionic acid, oxalic acid, glycolic acid, glyoxilic acid and glycine.

Other corrosive compounds are corresponding ammonium salts of the aforementioned acids which are obtained by reaction of the aforementioned acids with a primary, secondary tertiary amine present in the methods according to this invention, preferable the alkanolamine or its amines obtained by its conversion.

Corrosive compounds may also be carbamic acids. Carbamic acids may be obtained by the reaction of a primary, secondary or tertiary amine present in the methods according to this invention with carbon dioxide. Carbon dioxide may be formed by decarboxylation of alkanolamines, in particular MEOA, and its corresponding conversion products. Preferred carbamic acids are acids obtained by reaction of carbon dioxide with MEOA or the corresponding amines obtained by the conversion of MEOA.

Further corrosive compounds which may contribute to corrosion are carbamates. Preferably, the carbamates are formed by reaction of the aforementioned carbamic acid with a primary, secondary or tertiary amine present in the methods according to the present invention. In particularly, the carbamates are formed by reaction of an aforementioned carbamic acid with the alkanolamine, in particularly MEOA, or the amines obtained from its conversion. Surprisingly, it has been found that corrosion of stainless steel in the methods according to the present invention can occur at low concentrations of the aforementioned corrosive compounds. Accordingly, it is preferred to conduct the methods of the present inventions when the effluent of the methods according to the present invention comprises the aforementioned corrosive compounds, wherein the concentration of the aforementioned corrosive compounds in the effluent is in the range of 10 ppm to 500 ppm, preferably 20 ppm to 400 ppm, more preferably 30 to 350 ppm and most preferably 40 to 300 ppm.

### Process Specific Design

Considering water exposure, pressure exposure and temperature exposure, both manufacturing and separation equipment used in an Alkylene Oxide Process may comprise one or more exposed steel parts made of duplex steel in accordance with this invention. Manufacturing equipment items are usually not exposed to hydrogen since the process is usually conducted in the absence of additional hydrogen. It is therefore preferred, that at least one exposed steel parts of at least one manufacturing equipment item and of at least one separation equipment item are made of duplex steel in accordance with this invention.

This also applies to the Urea Process.

The Polyol Amination Process, in particularly the MEG Process, the DEG Process and the Polyglycol Process, as well as the Alkanolamine Conversion Process, in particularly the MEOA Process and the MPOA Process, as well as the Reductive Amination Processes, in particularly the GA Process, and as well as the Amino Acid Process are usually conducted in the presence of hydrogen. For these processes it is preferred, that only those steel parts of those equipment items which are not additionally exposed to hydrogen (within the meaning of the present invention) are made of duplex steel. Accordingly, it is preferred that exposed steel parts of equipment items which are additionally exposed to hydrogen, such as the reactor, flash vessels, heat exchangers, piping, valves, coolers which are located upstream of a step in which the hydrogen concentration is sufficiently reduced, are not made of duplex steel.

On the other hand, exposed steel parts of equipment items which are located downstream of one or more hydrogen separation steps in which the hydrogen concentration of the liquid phase is lowered to the hydrogen concentrations referred to above, are preferably made of duplex steel in accordance with this invention.

### Duplex Stainless Steel

According to the invention, one or more parts of one or more equipment item are made of duplex steel.

A duplex steel is a stainless steel comprising both an austenite and ferrite phase in its metallurgical structure.

The weight proportion of the austenite phase to the ferrite phase is preferably in the range from 30:70 to 70:30 and more preferably 40:60 to 60:40. Most preferably the phases are of roughly equal proportion.

Duplex steel preferably comprises Chromium in the range of 10 to 35 percent by weight and more preferably in the range of 20 to 30 percent by weight.

Duplex steel preferably comprises Molybdenum in a range of 0.1 to 5 percent by weight and more preferably 0.3 to 4 percent by weight.

Duplex steel preferably comprises Nickel in a range of 1 to 10 percent by weight and more preferably 1.5 to 7 percent by weight.

Duplex steel preferably also comprises Nitrogen in a range of 0.05 to 0.5 percent by weight and more preferably 0.1 to 0.4 percent by weight.

Preferably, duplex steel comprises more than one of above-mentioned elements in the afore-mentioned ranges.

More preferably, duplex steel comprises all off the above-mentioned elements in the afore-mentioned ranges.

In addition, duplex steels may comprise one or more of the following elements in the amounts specified below:
carbon, preferably in amounts of 0.05 percent by weight or less,
silicon, preferably in amounts of 1 percent by weight or less,
manganese, preferably in amounts of 10 percent by weight or less,
phosphor, preferably in amounts of 0.05 percent by weight or less,
sulfur, preferably in amounts of 0.03 percent by weight or less,
wolfram, preferably in amounts of 0.25 to 1.5 percent by weight,
cobalt, preferably in amounts of 0.25 to 3 percent by weight, 3275 copper, preferably in amounts of 0.05 to 5 percent by weight.

Preferred duplex steel are steels having the following UNS (Unified Numbering System) designation are selected from
S31200, S31260, S31803, S32001, S32003, S32101, S32202, S32205, S32304, S32506, S32520, S32550, S32750, S32760, S32808, S32900, S32906, S32950, S39274, S81921, S82011, S82012, S82013, S82031, S82122, S82441.

More preferred duplex steels are steels having the following UNS designation are selected from the group consisting of:
S31803 and S32750.

The scope present invention covers embodiments in which different exposed steel parts of an equipment item or different equipment items are made of more than one duplex steel grade.

However, it is preferred that all exposed steel parts of an equipment item which are made of duplex steel are made of the same duplex steel grade, to avoid residual risks resulting from using materials with slightly different properties.

The present invention is also directed to the use of duplex stainless steel as a construction material for parts of an equipment item used in an alkanolamine and/or alkyleneamine manufacturing process or separation process, which have one or more surfaces in contact with alkyleneamines and/or alkanolamines.

The present invention is also directed to the use of duplex stainless steel as a construction material for parts of a major equipment item and/or ancillary equipment item and/or fastening means used in an alkanolamine and/or alkyleneamine manufacturing process or separation process, which have one or more surfaces in contact with alkyleneamines and/or alkanolamines.

The present invention is also directed to the use of duplex stainless steel as a construction material for parts of a major equipment item, used in an alkanolamine and/or alkyleneamine manufacturing process or separation process, selected from the group of reactors, vessels, heat exchangers, condensers, agitators and columns, which have one or more surfaces in contact with alkyleneamines and/or alkanolamines.

The present invention is also directed to the use of duplex stainless steel as a construction material for parts of a ancillary equipment item, used in an alkanolamine and/or alkyleneamine manufacturing process or separation process, which have one or more surfaces in contact with alkyleneamines and/or alkanolamines, selected from the group of pumps, compressors, valves and pipes.

The present invention is also directed to the use of duplex stainless steel as a construction material for fastening means which have one more surfactes in contact with alkyleneamines and/or alkanoalimens, used in an alkanolamine and/or alkyleneamine manufacturing process or separation process, selected from the group of nuts, bolts, fittings, sealing rings, flanges and screws.

Further the present invention is also directed to the use of uplex stainless steel as a construction material for parts of an equipment item used in an alkanolamine and/or alkyleneamine manufacturing process or separation process, which have one or more surfaces in contact with alkyleneamines and/or alkanolamines, wherein the effluent from the alkanolamine manufacturing or alkanolamine conversion process comprises 10 to 500 ppm of a corrosive compound selected from the group consisting of of (i) acids, selected from the group consisting of formic acid, acetic acid, oxalic acid, glycolic acid, glyoxilic acid, propionic acid and glycine, (ii) the corresponding ammonium salts of a such acids, (iii) carbamic acids, and (iv) ammonium carbamates.

In addition the present invention is directed to the use of duplex stainless steel in a alkanolamines and/or a process for the conversion of alkanolamines wherein the alkanolamine manufacturing or alkanolamine conversion process conducted according to a at least one of the embodiments of the present invention, in particular according to a method according to at least one of the claims of the present invention.

The benefits of the present invention are that the reactors for an ethyleneamine and/or ethanolamine preparation process can be reduced with a thinner material thickness compared to conventional stainless steel. The reactors were also found to be more resistant to corrosion and wear. Accordingly, the service or maintenance intervals can be prolonged. Also, the the service life of the reactor is increased. The equipment items are less susceptible to damage. Accordingly, equipment failure occurs less often. Therefore, the capacity of a alkyleneamine and/or alkanolamine production process or an alkyleneamine and/or alkanolamine separation process can be increased due to longer uptimes in which product can be produced and/or separated.

The benefits of the invention are demonstrated by the following examples:

### COMPARATIVE EXAMPLE 1

In a plant for the manufacture of ethyleneamines from monethanolamine and ammonia, comprising the steps of (i) conversion of ammonia and monoethanolamine in the presence of hydrogen and an amination catalyst, (ii) a work-up section comprising a) a section for the removal of hydrogen and ammonia; b) a section for the removal of water; c) a section for the separation of value products comprising 1) a section for the separation of EDA and PIP from higher boiling components; 2) a section for the separation of EDA and PIP; 3) a section for the separation of MEOA and DETA from high boiling components and 4) a section for the separation of AEEA from high boiling components.

The section for the separation of value products c) comprised several distillation columns, which were either designed as conventional distillation columns (comprising a reboiler, a condenser, a stripping section and a rectifying section) or as a dividing wall column.

The appropriate separation equipment was made of stainless steel.

After a period of operation of 2 to 3 years from start-up, several parts of the section for the separation of value products (3) needed to be replaced due to significant corrosion. Especially the equipment in the sump of the appropriate columns used in sections 1), 2) and 3), such as the reboiler, the fittings and the pipes, and the internals, such as the column packing, showed signs of corrosions.

### EXAMPLE 1

The corroded equipment items of the plant described in Comparative Example 1 were replaced by appropriate equipment items made of Duplex steel. No substantial signs of corrosions were evident after more than seven years of operation form the exchange of the relevant equipment items.

Surprisingly, it was found that equipment items made of convention stainless steel in a plant for the manufacture of ethyleneamine are subject to substantial corrosion in a period of operation of less than three years. Replacement of these equipment items with equivalent equipment items made of Duplex steel resulted in an operation in which substantial signs of corrosion were not detected after more than seven years of operation from the exchange of the relevant equipment items.

1.-17. (canceled)

21. A process for the manufacture of alkanolamines and/or a process for the conversion of alkanolamines wherein the process is performed in one or equipment items, wherein one or more steel parts of the one or more equipment items, which have one or more surfaces in contact with an alkanolamine, are made of duplex steel, wherein the feature made of duplex steel means consisting of duplex steel.

22. The process according to claim 21, wherein the alkanolamine manufacturing process or the alkanolamine conversion process is a process selected from the group consisting of:
(i) a process for the manufacture of alkanolamines comprising the conversion of an alkylene oxide with an aminating agent (Alkylene Oxide Process);
(ii) a process for the manufacture of alkanolamines comprising the conversion of a polyol with an aminating agent (Polyol Amination Process);
(iii) a process for the manufacture of alkanolamines by conversion of hydroxy-aldehydes or hydroxy-ketones with an aminating agent (Reductive Amination Process);
(iv) a process for the manufacture of alkanolamines by hydrogenation of amino acids (Amino Acid Process);
(v) a process for the conversion of alkanolamines with an aminating agent (Alkanolamine Amination); and
(vi) a process for the conversion of alkanolamines, wherein an ethanolamine functional compound is converted with a primary amine in the presence of a carbon delivering agent.

23. The process according to claim 22, wherein the Polyol Amination Process is
(i) a process for the conversion of ethylene glycol with ammonia in the presence of an amination catalyst (MEG Process);
(ii) a process for the conversion of diethylene glycol with ammonia in the presence of an amination catalyst (DEG Process);
(iii) a process for the conversion of 1,2-propanediol or 1,3-propanediol with ammonia in the presence of an amination catalyst (PDO Process); or
(iv) a process for the conversion of polyglycols with ammonia in the presence of an amination catalyst (Polyglycol Amination), or
the Reductive Amination Process
(v) is a process for the conversion of glycolaldehyde with an aminating agent (GA Process); or
the Alkanolamine Amination Process
(vi) is a process for the conversion of monoethanolamine with ammonia in the presence of an amination catalyst (MEOA Process);
(vii) is a process for the conversion of 1,2-propanol amine or 1,3-propanol amine with ammonia in the presence of an amination catalyst (MPOA Process).

24. The process according to claim 21, wherein one or more steel parts of one or more major equipment item, which have a surface in contact with an alkanolamine, are made of duplex steel.

25. The process according to claim 24, wherein all steel parts of one or more major equipment items, which have one or more surfaces in contact with an alkanolamine, are made of duplex steel.

26. The process according to claim 24, wherein one or more steel parts of at least one or more ancillary equipment items, which have one or more surfaces in contact with an alkanolamine, is made of duplex steel.

27. The process according to claim 21, wherein all steel parts of all equipment items, which have one or more surfaces in contact with an alkanolamine, are made of duplex steel.

28. A process according to claim 21 wherein only those steels parts of all equipment items with one or more surfaces in contact with an alkanolamine are made of duplex steel which are in contact with a liquid phase comprising hydrogen, wherein the hydrogen concentration in the liquid phase is 0.1 weight percent or less.

29. The process according to claim 28, wherein
(i) in an Alkylene Oxide Process at least one steel part, which has one or more surfaces in contact with an alkanolamine, of at least one manufacturing equipment item and of at least one separation equipment item are is made of duplex steel, or
(ii) in an Alkanol Amination Process, an MEG Process, a DEG Process, a Polyglycol Amination Process, a GA Process, a Reductive Amination Process and an Amino Acid Process that the separation equipment downstream from one or more hydrogen separation steps, in which the hydrogen concentration of the liquid phase 1 is lowered to 0.1 weight percent or less.

30. The process according to claim 21, wherein (i) only those steel parts of one or more equipment items are made of duplex steel which are in contact with a liquid phase comprising alkanolamine and water, wherein the water concentration in the liquid phase is 10 percent by weight or more; or (ii) wherein only those steel parts of one or more equipment items are made of duplex steel which are in contact with a liquid phase, wherein the hydrogen concentration is 0.1 weigh percent or less.

31. The process according to claim 21, wherein the steel parts in contact with a allanolamine are additionally exposted to a temperature of 30° C. or more and/or a pressure of 10 mbar to 300 bar.

32. The process according to claim 21, wherein the weight proportion of the austenite phase to the ferrite phase in the duplex steel is in the range of 30:70 to 70:30.

33. The process according to claim 21, wherein the duplex steel comprises:
Chromium: 20 to 30 percent by weight;
Molybdenum: 0.1 to 5 percent by weight;
Nickel: 1 to 10 percent by weight; and
Nitrogen: 0.05 to 0.5 percent by weight.

34. The process according to claim 21, wherein the duplex steel is S31803 or S3270 according to the designation of the Unified Numbering System (UNS).

35. The process according to claim 21, wherein the reaction effluent obtained by a method according to claim 21 comprises at least one corrosive compound selected from the group consisting of (i) acids, selected from the group consisting of formic acid, acetic acid, oxalic acid, glycolic acid, glyoxilic acid, propionic acid and glycine, (ii) the corresponding ammonium salts of a such acids, (iii) carbamic acids, and (iv) ammonium carbamates.

36. The process according to claim 35, wherein the concentration of corrosive compounds is between 5 ppm and 500 ppm.

37. The process according to claim 21, wherein the process is carried out under conditions in which the concentration of each oxygen and carbon dioxide is 500 ppm or less.
